(19) United States
(12) Patent Application Publication
(10) Pub. No.: US 2009/0182578 A1
(43) Pub. Date: Jul. 16, 2009

(54) System and Method for Mental Health Disease management

(76) Inventor: Sam Ozersky, Toronto (CA)

Correspondence Address: RICHES, MCKENZIE & HERBERT, LLP, SUITE 1800, 2 BLOOR STREET EAST, TORONTO, ON, M4W 3J5, CA

(21) Appl. No.: 11/921,394

(22) PCT Filed: May 23, 2006
(86) PCT No.: PCT/CA2006/000835

§ 371 Date: Nov. 30, 2007

Related U.S. Application Data
(60) Provisional application No. 60/685,793, filed on Jun. 2, 2005.

(51) Int. Cl. G06Q 50/00; A61B 5/00; G06Q 99/00; G06Q 40/00

(52) U.S. Cl. 705/3; 600/300; 705/2; 705/10; 705/4

(57) ABSTRACT

The present invention is a system and method of related healthcare processes protecting workplace productivity and mental health. It provides tools to patients and their physicians to assist them in identifying, managing and tracking mental health disorders. It also provides information to employers and health and disability insurers regarding the most effective interventions promoting productivity and mental and physical health. Through an interactive process with a website, the patient undergoes an initial screening for the presence of a mental health disorder, in an anonymous and confidential manner. After the first stage of screening, if the possibility of a mental disorder is identified, questions will be posed to the patient to determine the level of risk for possible diagnosis of a mental disorder by the M.D. A printable diagnostic risk map is provided in addition to appropriate care maps and follow up maps that guide physician and patient management of these disorders. Compliance with current best practices in disease management by both patient and physician is supported by scientific information at the lay level and physician level. Clinical and functional outcomes are quantified economically. Outcomes are tracked individually, but only aggregate information is provided to the employer and/or insurer. The overall result is improved mental health, physical health, productivity for large populations in a cost effective manner without requiring changes to the existing health care systems

### SCOPE OF THE INVENTION

The present invention relates to a system and a method for management of mental health disease which results in improved mental health of the patient and reduced economic loss in the workplace due to mental health related disability and productivity loss.

### BACKGROUND OF THE INVENTION

The World Health Organization released a statement in 2001 that mental health disorders rank first among all diseases in terms of causing disability in the United States, Canada and Western Europe, accounting for 25% of all disabilities. The American Medical Association estimates that, in 2002, there were 17.5 million American adults, or 8.3% of the adult population, with serious mental health disorders. Rates of serious mental health disorders were highest for persons aged 18 to 25 at 13%, which is the age at which most people are beginning their working careers. The most common form of mental health disorder is depression, which represents 50% of all mental health disorders, followed by anxiety disorders at 25%, and followed by substance abuse at 15%.

Early diagnosis and proper treatment are two of the key factors for successful recoveries from mental health disorders. If a mental health disorder is left untreated in the first six months, it often leads to long-term mental health problems. However, only 12 in 100 patients with mental health disorders are properly diagnosed and treated according to guideline practice levels of the psychiatric industry, also known as the “best practices”.

Early diagnosis is difficult because a patient must first realize that he or she may have a mental health disorder before he or she seeks professional diagnosis and treatment. However, patients often confuse a mental health disorder, such as depression, with less serious conditions, such as unhappiness, stress or burnout, and subsequently do not seek professional help. As another example, 75% of alcoholics do not believe that they have a drinking problem, and therefore, avoid seeking professional help.

Furthermore, proper treatment is difficult because it first requires a correct diagnosis. However, correct diagnosis of mental health disorders is difficult in the context of the current mental health industry. As of 2000, there were over 598,000 registered physicians and surgeons in the United States, of which only 33,500 or 5.6% were psychiatrists. There were also an estimated equal number of psychologists that provide mental health treatments. Therefore, there were only about 20 psychiatrists and psychologists per 100,000 population. Patients are typically referred to psychiatrists or psychologists by their family physicians, and because of the significant shortage of mental health professionals in North America, many are treated in a primary care setting by non specialists who often spend a maximum of 15 minutes on an assessment that a psychiatrist would take an hour to perform. Depending on the geographic area, there can be a long waiting time to see a psychiatrist. The long delay before an initial consultation with a psychiatrist or psychologist makes early diagnosis and treatment unlikely, and is a considerable hindrance to the recovery time of a mental health patient.

As a result of the above-mentioned shortage of mental health professionals, at least 75% of all mental health care is actually delivered by family physicians. However, family physicians do not have the specialized training of psychiatrists and psychologists, and cannot be expected to keep up-to-date with the most recent advances in therapy and drug administration for mental health disorders. Furthermore, whereas psychiatrists typically spend an hour per patient visit, family physicians spend only about 15 minutes per visit. Therefore, proper diagnosis and treatment at the “best practices” level cannot reasonably be expected to be delivered by family physicians.

Mental health disorders have had an especially negative effect in the workplace. One in five employees suffers from a mental disorder for an extended period of time during their careers. Further, the rate of mental health disorders is increasing the most for younger persons early in their careers.

Mental health disorders are the greatest cause of: (i) worker absenteeism; and (ii) productivity loss in the workplace. Health Canada estimates that mental health problems cause Canadian business $30 billion annually. An Ontario study concludes that worker absenteeism from addiction problems alone cost corporations $1.5 billion per year. In the U.S., estimates vary between $150 billion to $300 billion lost annually by businesses due to mental health problems.

Many employees do not seek medical help even when they suspect that they have a mental health disorder because they are concerned with their reputation in the workplace and because they fear losing their jobs. This especially applies to employees who are in higher and/or more senior positions. Most employees also confuse a mental health disorder with a less serious condition, such as unhappiness, stress or temporary burnout, and believe these conditions will pass. However, many mental health disorders require actual medical treatment for there to be a successful recovery. Furthermore, proper and early treatment leads to a quicker recovery. For example, with respect to depression, treatments which are guided by a “best practices” protocol result in a 70% recovery rate within seven months, in comparison to a 20% recovery rate with the usual family physician treatments. Employee Assistance Programs (EAP) are often set up to help employees resolve workplace issues, such as manager-employee problems, skills counselling, and personal crises, including mental health concerns. Typically, an employee can telephone a hotline to receive basic counselling for a limited number of sessions. However, such programs do not adequately address the need for early diagnosis and proper treatment of mental disorders because: (a) the employee may not realize he or she has a mental health disorder and therefore, will not telephone the hotline; and (b) the counsellors are generally not psychiatrists and nor are they qualified to make a diagnosis. They deal with stress and distress, not medical illness of which mental disorders are a subset.

Systems for the self-identification and screening of mental health problems are known. For example, there have been widespread community screening of mental health disorders through the newspaper and self-screening programs on the Internet. There are internet websites which provide questionnaires to screen patients for mental health disorders.

Further, systems for diagnosis of mental health disorders are also known. There are interactive websites which analyze a user's answers, and provide either a diagnosis or information to assist a physician in making a diagnosis.

However, previous systems for identifying mental health disorders focus on only one or two specific aspects required for the successful recovery of a patient. In order to promote a positive outcome in the, management and tracking of a mental health disorder, there must be each of the following components:

(a) self-identification by the patient;

(b) early and correct diagnosis by the physician;

(c) proper treatment of the mental health disorder; and

(d) follow-up to ensure compliance with the treatment.

WO 01/69513 to Zakim teaches a computer-based system and method which constructs medical histories by direct interactions between the patient and the system and which requires pertinent and relevant medical information covering the complete life of a given patient. However, the Zakim system does not teach all of the above-mentioned necessary components in order to produce a positive outcome in the management and tracking of a mental health disorder. As mentioned above, two of the key factors for successful recovery from mental health disorders are early diagnosis and proper treatment. One of the problems associated with mental health disorders is that a patient must first realize that he or she has a mental health disorder before he or she seeks professional diagnosis and treatment. However, people often confuse a mental health disorder, such as depression, with a less serious condition, such as unhappiness, stress or burnout, and believe that these conditions will pass. Subsequently, they do not seek professional help. As such, the problems of identifying mental health disorders and getting the patient to a doctor's office are very specific to mental health disorders.

The Zakim system fails to provide any mechanism to identify patients who are likely to have a mental health disorder in cases where the patient himself or herself does not realize he or she has a mental health disorder. At page 12, lines 11 to 18 of Zakim, it is taught that the system and method of this reference involves taking the medical history of a patient in a manner which is akin to one taken by an expert physician. At page 23, lines 4 to 9, Zakim teaches that the first question asked of the patient is to describe why medical assistance is sought. This description is meant to be in the patient's own words and obtained without prompting by the physician. This is called the “chief complaint”. In the system of Zakim, the patient types in their chief complaint when prompted by a computer screen. In the system and method of Zakim, the patient clearly must first realize that he or she has a health problem prior to using the system and method. Further, in the system and method of Zakim, the patient who realizes that he or she has a health problem must also seek medical assistance and describe their chief complaint to the system. The Zakim system, therefore, fails to assist the patient in first realizing that he or she has a health problem.

Another problem specific to mental health disorders is getting a patient to go to a doctor's office in order to obtain the diagnosis, treatment and follow-up that he or she needs. The system of Zakim fails to overcome this problem because it only begins operating when the patient arrives at the doctor's office. Specifically, the Zakim system fails to assist the patient in recognizing that he or she has a mental health problem and fails to encourage the patient to go to a doctor's office. In the system and method of Zakim, a large number of patients who have hidden diagnosable mental health disorders would not be identified.

In addition, the system and method of Zakim is designed for patients with physical health disorders, and not patients with mental health disorders. Zakim teaches rule-based searching of the input data in the medical history for predetermined quality of pain words and phrases, matching identified quality of pain words and phrases with the selected set of preliminary working diagnosis. Thus, the system and method of Zakim focuses on physical health disorders where the patient feels pain, and as a result of the pain, realizes he or she has a health problem and subsequently seeks medical assistance. Physical health disorders begin with a physical complaint which is easy for the patient to recognize. For example, in a physical health disorder, the patient may feel pain, be short of breath, have a rash, have constipation, experience vomiting or the like.

In contrast, in a mental health disorder, the patient may not feel physical pain and therefore, would not seek medical assistance. Mental health disorders do not have the physical complaints which are associated with physical health disorders. In fact, many patients with mental health disorders confuse their symptoms with the normal up and downs and stresses of daily living. For example, many people believe that the anxiety they feel is merely stress related to workplace conditions, such as a difficult employer or supervisor. This anxiety, however, may be a signal symptom of a major depressive episode and a significant and treatable medical condition.

A specific example is social anxiety disorder which is a psychiatric condition causing an avoidance of social contact because of a fear of humiliation or shame. Social anxiety disorder is often confused with a person who is merely shy. This disorder often leads to social withdrawal and a reduced ability to have relationships, as well as a reduced ability to succeed in the workplace. Social anxiety disorder responds well to medications in the class of fluoxitene (Prozac). However, the system and method of Zakim depends on the patient identifying physical complaints which are not present in mental health disorders such as social anxiety disorder. As such, mental health disorders, such as social anxiety disorder, would not be identified by the Zakim system.

Another problem specifically associated with mental health disorders is that patients do not seek medical help because of the stigma attached to mental health symptoms such as sadness, panic attacks, fears, and binge eating, and these patients fear losing respect in their personal, social and workplace environments. Mental health disorders cause particular problems for employees in the workplace because, they are concerned with their reputation in the workplace. This especially applies to employees who are in higher and/or more senior positions. Many employees do not seek medical help or mental health counselling through employee assistance plans even when they suspect they have a mental health disorder because they must reveal their identity and they fear information regarding their mental health will be made known to others in their workplace.

The confidentiality offered by previous systems such as Zakim is not sufficient to overcome this problem. Confidentiality information is not completely secure from being accessed by third parties. For example, if a patient's confidential mental health information is stored in a data base, a skilled computer hacker could access such information. As a result, even if a patient knows that his or her mental health information will be stored in a confidential database, he or she will not consider this sufficient protection, and he or she will not seek medical assistance for fear this information will be made known to others.

A further problem associated with mental health disorders is that many patients will not consider going to their doctor unless the problem is serious and treatable, because they fear that it will cause their premiums to go up or they will be denied coverage for attending a doctor's office with a complaint related to mental health. A patient is more motivated to spend money to go to a doctor's office when he or she believes that there is a serious risk for a mental health disorder and that this disorder is treatable.

In the United States, one out of three individuals does not have medical insurance because they cannot afford the premiums and they do not qualify for government assistance. If such individuals go to visit the doctor, they must pay from their own pocket for this visit. Such individuals are reluctant to spend money for a visit to the doctor for what they consider to be an insignificant complaint. However, as mentioned above, the symptoms of a mental health disorder often do not involve the physical complaints of a physical health disorder. As such, a patient may dismiss their mental health symptoms as insignificant complaints for which they do not want to spend money. The Zakim system fails to overcome this problem because, until the patient attends his or her doctor's office, there is no mechanism to make a patient aware that he or she may have a mental health problem that is both serious and treatable.

In addition, the Zakim system fails to provide an effective mechanism of follow-up to ensure compliance with the treatment. The system of Zakim does not inform the patient as to whether his or her mental health disorder is getting better or worse. If the patient is informed that his or her condition is deteriorating and that a treatment is not effective, he or she is more likely to go to the doctor for a follow-up visit. Zakim fails to provide such information to the patient. As another example, with anti-depressant treatment, it is important to complete at least six months of treatment, even if the patient feels as if he or she has recovered in one month. A typical uninformed patient will stop the treatment too soon. Although the patient may feel as if he or she has recovered prior to six months, such patients have a high probability for relapse and could possibly be unresponsive to the same medication. Family doctors rarely have the time to explain to a patient the risk of early treatment cessation and to provide easily understood scientific backup information. The system of Zakim does not include any specifically designed educational material for the patient to enhance compliance.

U.S. Patent Application Publication No. 2003/0149596 A1 to Bost teaches a system and method for measuring the relative economic benefits to employers for services offered by healthcare plans. The system of Bost calculates a monetary evaluation to the employer of the reduction in the absenteeism and low productivity days based on the firm's average revenue per employee, average daily wage, and other parameters. Bost measures the value of the health plan by determining how much a particular health plan changes the absenteeism and lost productivity due to, for example, depression.

However, the system of Bost is not specially adapted for mental health disorders. First, Bost fails to recognize the problem associated with mental health disorders in that people must first realize that they have a mental health disorder. Bost further fails to recognize the problem that in the workplace, employees who suspect that they might have a mental health disorder are reluctant to seek medical assistance.

It is therefore an object of the present invention to provide a novel business tool useful to reduce economic loss in the workplace due to mental health related disability and productivity loss.

It is an object of the present invention to provide a system and method which integrates all of these components which are required to promote positive outcomes in the end to end identification, management and tracking of mental health disorders utilizing a patient driven Web based interactive system.

It is an object of the present invention to provide a system and method which facilitates early and correct diagnosis of the mental health disorder by the physician. If, for example, depression is not detected within the first six months, it is likely to become chronic. Further, family physicians, who provide approximately 75% of all mental health care, lack the proper training and time to provide a correct diagnosis of a mental health disorder.

It is an object of the present invention to provide a system and method which facilitates the proper and timely treatment of mental health disorders without the requirement of specialist intervention. Many mental health disorders require the proper medical treatment in order to promote a successful recovery. Furthermore, the proper medical treatment always results in a faster recovery. For example, in depression, when treatments are made in accordance with “best practice” guidelines, 70% recover within seven months, as opposed to only a 20% recovery rate for the usual family physician treatments.

It is an object of the present invention to provide a system and method which facilitates follow-up of the medical treatment of mental health disorders. It is known that over 50% of patients do not comply with treatment guidelines. The rate of compliance with Best Practices by primary care physicians is less than 50%. The combination of patient and physician factors results in rates of effective management of mental disorders in primary care of only 1 in 8 cases. Proper follow-up guided by Best Practice protocol for treatment (called CARE MAPS) which is shared and available to the patient and the physician, and encourages adherence and compliance with “best practices” treatment by both parties. It is also an object of the present invention to provide a system that facilitates simultaneous compliance with Best Practices by both key parties. If either the patient or the physician is remiss is adhering to Best Practices, the other party points to the same CARE MAP to encourage adherence and compliance.

It is an object of the present invention to provide a system and method which facilitates follow-up of the medical treatment of mental health disorders. It is known that over 50% of patients do not comply with treatment guidelines. The rate of compliance with Best Practices by primary care physicians is less than 50%. The combination of patient and physician factors results in rates of effective management of mental disorders in primary care of only 1 in 8 cases. Proper follow-up guided by Best Practice protocol for treatment (called CARE MAPS) which is shared and available to the patient and the physician, and encourages adherence and compliance with “best practices” treatment by both parties. It is also an object of the present invention to provide a system that facilitates simultaneous compliance with Best Practices by both key parties. If either the patient or the physician is remiss is adhering to Best Practices, the other party points to the same CARE MAP to encourage adherence and compliance.

Furthermore it is an object of the present invention to provide a “follow-up function” that monitors and tracks clinical and functional outcomes in relation to treatment in order to determine if a change in treatment is required.

It is also an object of the present invention to provide a system and method by which the patient can anonymously determine whether he or she may be at risk for mental health disorder. Mental diagnosis can only be made by a physician.

It is also an object of the present invention to provide a system and method where a patient can conveniently access detailed medical information with respect to his or her mental health disorders.

It is also an object of the present invention to provide a system and method to reduce workplace absenteeism, and short term and long term disability, and to determine and quantify with respect to days and dollars the extent of loss prevented. As such it is a system and method that improves human productivity and return on investment in any business relying on participation of human labour.

### SUMMARY OF THE INVENTION

The present invention is a system and method of related healthcare processes protecting mental health and workplace productivity. It provides tools to patients to assist them and their physicians in identifying, managing and tracking mental health disorders. Through an interactive process with a website, the patient undergoes an initial screening for the presence of a mental health disorder, preferably in a confidential manner. After the first stage of screening, if the possibility of a mental disorder is identified, questions will be posed to the patient to determine if he or she is at high risk for a mental disorder diagnosis by a physician. This is followed by provision of Care Maps and Follow up Maps to guide compliance with best practices by both patient and physician.

In one aspect, the present invention provides a method for treating a mental health disorder and improving workplace productivity and disability reduction related to said disorders comprising the steps of: (a) having a patient access an interactive website for an initial visit; (b) having the patient answer one or more screening questions to determine whether there is a possibility of the patient having a mental health disorder; (c) processing the patient's answers to the one or more screening questions to determine whether there is the possibility of the patient having a mental health disorder; (d) where there is the possibility of the patient having a mental health disorder, selecting a module of diagnostic risk assessment questions related to a specific mental health disorder; (e) having the patient answer the diagnostic risk assessment questions related to the specific mental health disorder; (f) processing the patient's answers to the diagnostic risk assessment questions to determine the level of risk for a particular diagnosis; (g) providing the patient with a preliminary diagnostic risk assessment map which comprises a list of the patient's answers to the screening questions and the diagnostic risk assessment questions, and provides the level of risk for a particular diagnosis; (h) providing the patient with a CARE MAP which comprises evidence-based instructions for patients and physicians on possible treatments; (i) providing the patient with a follow-up map which comprises data on symptom and functional impairment severity trends over time, absenteeism, presenteeism, and medical and personal history of the patient, as provided by the patient; (j) having the patient share the preliminary diagnostic risk assessment map, the CARE MAP and the follow-up map with a physician in printable form or via electronic records; (k) after considering the preliminary diagnostic risk map, the CARE MAP and the follow-up map, the physician instituting medical treatment of the patient; (l) providing the patient with a follow-up reminder via the internet and/or telephone to access the interactive website for a return visit; (m) having the patient access the website for the return visit; (n) having the patient answer the diagnostic risk questions from step (e) again; (o) having the patient answer questions regarding management history of the medical treatment; and (p) processing the patient's answers to the questions from steps (n) and (o) to determine whether there should be a change in the medical treatment of the patient.

A system for treating mental health disorders and improving workplace productivity and disability reduction related to said disorders, wherein said system comprises an interactive website, said website comprising: (a) a web page for accessing the web site; (b) a screening questionnaire for determining whether there is a possibility of a patient having a mental health disorder; (c) means for processing the answers to the screening questionnaire to determine whether there is the possibility of the patient having a mental health disorder; (d) a diagnostic questionnaire for determining the patient's level of risk for a particular diagnosis; (e) means for processing the answers to the diagnostic questionnaire to determine the patient's level of risk for a particular diagnosis; (f) means for providing a preliminary diagnostic risk assessment map which comprises a list of the patient's answers to the screening questionnaire and the diagnostic risk assessment questionnaire, and provides the level of risk for a particular diagnosis; (g) means for providing a CARE MAP which comprises evidence-based instructions for patients and physicians on possible treatments; (h) means for providing a follow-up map which comprises data on symptom/functional impairment trends over time, and medical and personal history of the patient, as provided by the patient; and (i) following medical treatment, means for providing a follow-up reminder via the Web and/or phone to the patient to access the website for a return visit.

In another aspect, the present invention provides a method for providing aggregate economic information to the employer/insurer regarding productivity losses due to mental disorders, most effective medical interventions in terms restoration of productivity level and reduction of disability and aggregate information to public and private health insurers on most effective medical interventions that limit morbidity and mortality and promote early remission and recovery, said method comprising: (a) having a patient access an interactive website for an initial visit; (b) having the patient answer one or more screening questions to determine whether there is a possibility of the patient having a mental health disorder; (c) processing the patient's answers to the one or more screening questions to determine whether there is the possibility of the patient having a mental health disorder; (d) where there is the possibility of the patient having a mental health disorder, selecting a module of diagnostic risk assessment questions related to a specific mental health disorder; (e) having the patient answer the diagnostic risk assessment questions related to the specific mental health disorder; (f) processing the patient's answers to the diagnostic risk assessment questions to determine the level of risk for a particular diagnosis; (g) providing the patient with a preliminary diagnostic risk assessment map which comprises a list of the patient's answers to the screening questions and the diagnostic risk assessment questions, and provides the level of risk for a particular diagnosis; (h) providing the patient with a CARE MAP which comprises evidence-based instructions for patients and physicians on possible treatments; (i) providing the patient with a follow-up map which comprises data on symptom and functional impairment severity trends over time, and medical and personal history of the patient, as provided by the patient; (j) having the patient share the preliminary diagnostic risk assessment map, the CARE MAP and the follow-up map with a physician in printable form or via electronic records; (k) after considering the preliminary diagnostic risk map, the CARE MAP and the follow-up map, the physician instituting medical treatment of the patient; (l) providing the patient with a follow-up reminder via the internet and/or telephone to access the interactive website for a return visit; (m) having the patient access the website for the return visit; (n) having the patient answer the diagnostic risk questions from step (e) again; (o) having the patient answer questions regarding management history of the medical treatment; (p) processing the patient's answers to the questions from steps (n) and (o) to determine whether there should be a change in the medical treatment of the patient; (q) having the patient answer questions about job classification, days absent, salary per day, days present but having percent productivity loss, days with symptoms, days of treatment (type and dosage), said data collected allowing correlation between clinical status, treatment, and workplace costs and savings (clinical economic correlation); (r) processing data from (q) using analytical tools to derive clinico-economic information; and (s) reporting to an entity arranging for access to the website with aggregate information regarding the value of workplace economic loss and benefit of the method.

Advantageously, the system and method of the present invention links together all of the required steps for a successful recovery from a mental health disorder, specifically: (i) self-identification by the patient; (ii) early and correct diagnosis by the physician; (iii) proper treatment of the mental health disorder; and (iv) follow-up to ensure patient and physician compliance with Best Practices.

The system and method of the present invention potentially improves recovery rates from mental health disorders by up to 300% over average existing symptoms and methods treatments.

Advantageously, the system and method improves outcomes, reduces public and private insurer health care costs and reduces the incidence and prevalence of physical disorders. For example a patient who has had a heart attack is five times as likely to die if he or she suffers from concurrent depression. Depression is a risk factor for the development of coronary artery disease and untreated depression causes loss of brain tissue visible on MRI scanning.

Further, the system and method of the present invention is preferably designed such that the patient can access the website anonymously.

Also, the system preferably provides the patient with access to medical information concerning mental health disorders in lay terms. It has been found that providing a patient and physician with an explanation of the clinical and/or medical science behind his or her mental health disorder can be effective in boosting patient and physician compliance with taking and prescribing Best Practice treatment.

Advantageously, the cost to an employer for implementing the system of the present invention in the workplace is relatively low, about $3.00 per year per employee. Employers will recover these costs if the system of the present invention reduces worker absenteeism by only 1% or if it eliminates 0.1% of all disability claims. It is expected that the return on investment for the employer will be in the order of 3:1 to a possible 20:1 depending on how effectively the system is promoted to employees and health care providers. Also, the system of the present invention will benefit employers by improving worker morale and productivity. In addition, the system of the present invention can either stand alone or easily be integrated with an existing employment assistance program (EAP) or disability programs. Advantageously, the system and method of the present invention will dramatically improve the effectiveness of family physicians. The system and method will help to save family physicians valuable time required in making a correct diagnosis and in prescribing the proper treatment and follow-up. The system of the present invention is a virtual psychiatrist consultant to the patient and the family physician, and gives them the knowledge and tools to obtain the best clinical and functional outcomes.

Advantageously, the system and method of the present invention will reduce existing costs for insurance companies by reducing disability incidences and the duration of such incidences. Also, the system and method of the present invention supports decision-making in claims management, thus reducing the need for independent medical examinations and prolonged short term disability/long term disability.

Advantageously, the system of the present invention provides new distribution channels for drug descriptions and clinical information. The system and method of the present invention can also be used by pharmaceutical companies as a source of finding candidates for clinical trials.

A further advantage of the system and method of the present invention is that it helps a patient to realize that he or she has a mental health disorder and encourages him or her to go to the doctor's office. This is in contrast to previous systems such as that of Zakim which does not assist the patient in seeking a mental health assessment and medical treatment. The system of the present invention bridges the gap between patients and their doctor. The method of the present invention includes a step of having the patient answer one or more screening questions to determine whether there is a possibility of the patient having a mental health disorder. These screening questions identify patients who are likely to have a mental health disorder, and this includes patients who do not realize themselves that they have a mental health disorder. Such patients would, otherwise, be unlikely to seek the medical assistance that they need.

In addition, the system and method of the present invention is designed specifically for patients with mental health disorders. Previous systems such as Zakim are inherently directed to treating patients with physical health disorders. Such systems require the patient to experience a physical complaint, such as a rash or pain, prior to seeking medical assistance. These previous systems are not effective for mental health disorders which often do not involve physical complaints. For example, social anxiety disorder does not involve a physical complaint and is often confused with merely being shy. The system of the present invention is specifically designed for patients with mental health disorders by helping the patient to realize that a non-physical symptom warrants a visit to his or her doctor's office.

Advantageously, the system and method of the present invention preferably allows the patient to access the web site anonymously. For example, the patient is provided with a user I.D. and passport for accessing the website. Mental health disorders have a stigma attached to them such that many people will not seek medical health or mental health counselling even when they suspect they have a mental health disorder because they must reveal their identity and they fear that information regarding their mental health will become known to others in their family, social, and workplace environments. Previous systems such as Zakim aim to keep such mental health information confidential, such as by storing such information in a confidential database. However, such confidential information is not completely secure from being accessed by third parties. For example, a computer hacker could access a confidential database with the patient's mental health information. In contrast, the anonymous access feature of the present invention allows the patient to use the system and method of the present invention without any fear of being identified as having a mental health disorder in his or her family, social and workplace environments. Specifically, the patient has the opportunity to determine whether or not she is at risk of having a mental health disorder under the security of anonymity.

Another advantage of the system and method of the present invention is that it overcomes a problem where a patient is reluctant to spend money for medical treatment until he or she recognizes that he or she is at serious risk of having a mental health disorder and that this mental health disorder is treatable. This problem cannot be overcome by previous systems such as Zakim which are not effective until the patient actually goes to see his or her doctor. In the system and method of the present invention, prior to seeing his or her doctor, the patient answers diagnostic risk assessment questions and is provided with a preliminary diagnostic risk assessment map which indicates the level of risk for a particular diagnosis. Further, the system and method of the present invention provide the patient with a follow-up map which comprises data on symptom and functional impairment severity trends over time. As such, a patient will be able to recognize that he or she is at serious risk for a mental health disorder and that this disorder is treatable and therefore, would be motivated to spend the money associated with visiting his or her doctor.

The system and method of the present invention also includes, subsequent to visiting the doctor and getting medical treatment, a return visit to the website where the patient answers the diagnostic risk questions again, as well as questions regarding management history of the medical treatment. The website processes the patient's answers and determines whether there should be a change in the medical treatment of the patient. The system and method of the present invention advises the patient as to whether or not his or her mental condition is getting better or worse. The patient learns whether or not the medical treatment that has been instituted is effective. If the patient learns that the medical treatment is not effective, he or she is more likely to go back to the doctor for a follow-up visit and receive an alternative treatment. As well, the website of the present invention provides hyperlinks to literature on the clinical and/or medical sciences of mental health disorders. This literature includes, for example, the risks of ceasing treatment early, with easy to understand scientific backup information. A common problem with treating mental health disorders is that a patient will stop their treatment too soon. For example, antidepressant treatment generally requires at least six months of treatment even though the patient may feel as if he or she has recovered after one month. If the patient ceases treatment too soon, there is a high probability of relapse and the patient could possibly be unresponsive to the same medication. The system and method of the present invention provides literature to the patient which explains the risks of early treatment cessation and therefore discourages patients from doing so. Previous systems such as Zakim do not provide such specifically designed educational materials for the patient to enhance compliance.

The system and method of the present invention provides aggregate economic information to the employer/insurer regarding the productivity losses due to mental disorders, most effective medical interventions in terms of restoration of productivity level and reduction of disability and aggregate information to public and private health insurers on most effective medical interventions that limit morbidity and mortality and promote early remission and recovery. Therefore, the system and method of the present invention has the advantage of linking specific medical treatments to their impact on the time for a patient to return to the workplace, thus being useful as a business tool. For example, the system and method of the present invention can determine which one of two different drugs used for treating depression, Effexor and Prozac, are more effective in reducing the time it takes for patients to return to work. Previous systems such as that of Bost do not provide any data regarding the link between specific medical treatments and workplace productivity.

### BRIEF DESCRIPTION OF THE DRAWINGS

Further aspects and advantages will become apparent from the following description taken together with the accompanying drawings in which:

FIG. 1 is a flow chart outlining method steps in the method of the present invention;

FIG. 2 is a graph illustrating the improved recovery rate after seven months when using the system and method of the present invention over current systems (protocols or “Care Maps”) and methods for treating mental health disorders;

FIG. 3 is a flow chart illustrating various stages of the method of the present invention during an initial visit by the patient; and

FIG. 4 is a flow chart illustrating various stages of the method of the present invention during a return visit by the patient.

### DESCRIPTION OF THE PREFERRED EMBODIMENTS

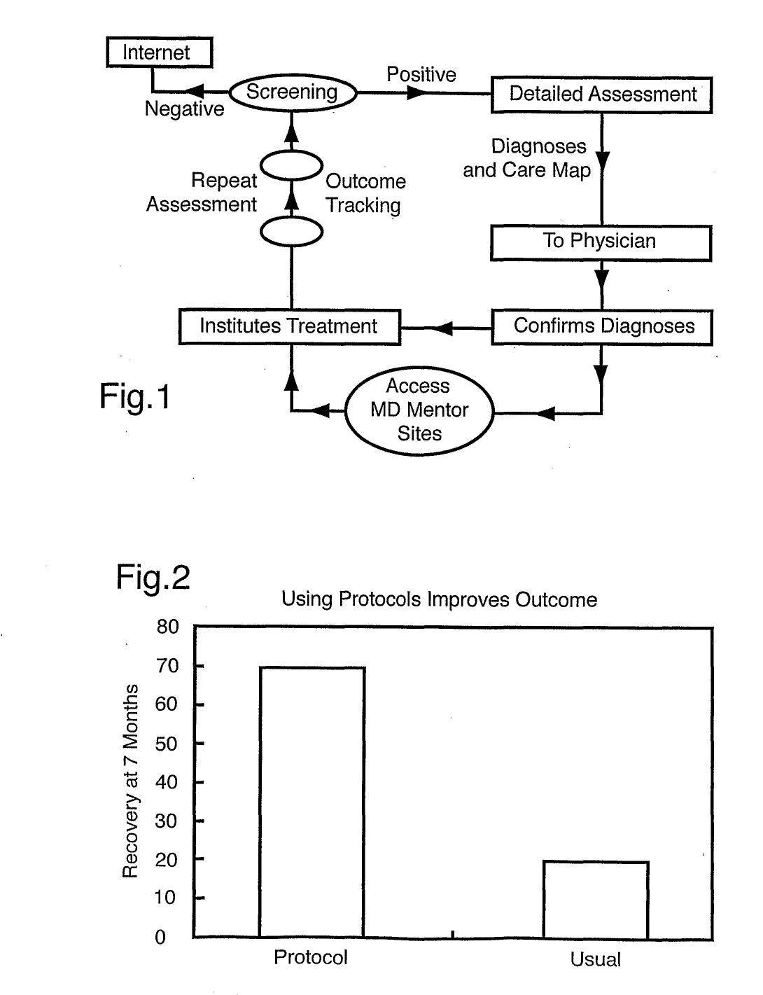

FIG. 1 illustrates an overview of the system and method of the present invention. The patient is asked basic screening questions to determine if, how many, and which further questions need to be asked to ascertain the presence or absence of a mental health disorder. If the answers to the basic screening questions show that there is the possibility of a mental health disorder, then a more detailed assessment is made. Specific questions are asked pertaining to an appropriate diagnostic risk module, wherein each diagnostic risk module relates to a specific mental health disorder such as depression, bipolar disorder, post traumatic stress disorder, generalized anxiety disorder, panic anxiety disorder, obsessive-compulsive disorder, and alcohol dependency. The patient's answers are processed, and the system provides the patient with a personalized preliminary diagnostic risk map, a care map and a follow-up map.

If the diagnosis risk assessment suggests the presence of a mental health disorder, the patient is encouraged to share the maps with his or her family physician. The preliminary diagnostic risk map lists the patient's answers to the specific diagnostic risk questions, and saves the physician considerable time and effort in making a proper diagnosis and prognosis. The care map provides the doctor with user-friendly guidelines to administer expert medical care, ensuring that the proper psychotherapy and/or medication is administered for the appropriate length of time. The patient having a copy of the care map is informed of what the best practices in treatment are and can determine if the best possible care is being delivered. Availability of the care map to the patient encourages the patient to follow the best practices without the necessity of the physician explaining what they are (care maps) and why they should be followed. (Tell Me More buttons that explain the scientific evidence in lay language why the Care Maps should be followed)

The physician examines the patient in the usual manner and may confirm/deny the preliminary diagnostic risk assessment provided by the diagnostic risk map generated by the system of the present invention. If desired, the physician can access the website which provides MD Mentor sites with literature and continuing medical education on relevant clinical and/or medical science relating to the specific mental health disorder diagnosed by the physician.

With assistance from the preliminary diagnostic risk map, the care map and possibly additional clinical and/or medical science information from the website, the physician institutes medical treatment of the patient.

The patient receives regular follow-up e-mails or phone calls every 3 weeks, and the follow-up map monitors treatment compliance, clinical condition change and functional changes in home life, health and workplace, including productivity.

As shown in FIG. 2, the system and method of the present invention (Care Maps are equivalent to Protocols in the study) improves the rate of recovery at seven months by about 300% over current systems and methods for treating mental health disorders.

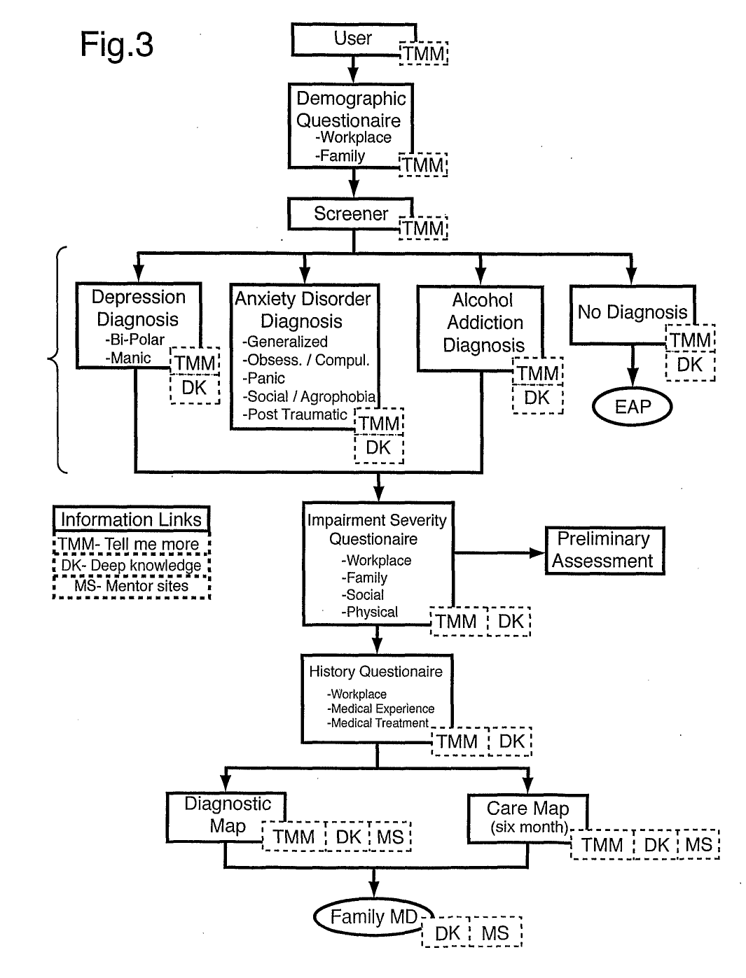

FIG. 3 provides a detailed illustration of the various stages of a patient's initial visit to the website. The patient is given an access code in order to access the website with full confidentiality and anonymity. He or she is then asked to generate a nickname and also an alias so that there is no chance of personal identification.

The patient fills out a demographic questionnaire. The information is anonymous and general, and the patient is not asked to provide his or her name. Some of the questions are required to be answered, such as age, sex and family history of health disorders. Other questions are optional, such as geographical location, length of employment, and number of workdays missed.

A screener test of 20 questions determines if, how many, and which further questions need to be asked to ascertain the presence or absence of a mental health disorder. If the answers to the screener test point to no clinical diagnosis, the patient is directed to an exit page. If there is the possibility of a mental health disorder, the patient is directed to the appropriate diagnostic risk module of questions.

In FIG. 3, the specific diagnostic risk modules are: (i) depression, and bipolar disorder; (ii) anxiety disorders, including generalized anxiety disorder, obsessive-compulsive disorder, panic, social anxiety disorder, and post-stress disorder; obsessive compulsive disorder and (iii) alcohol dependency. The system may include other disorders including child and adolescent disorders, geriatric disorders and eating disorders not shown in FIG. 3.

The specific diagnostic modules provide a series of questions in a sequence that is dynamically determined by answers to previous questions. Answers may be in terms of yes/no and/or a rating scale. The nature and order of the questions will be determined by the diagnostic rules for the test.

After processing the patient's answers, the system generates a preliminary diagnostic risk map, a care map and a follow-up map. The preliminary diagnostic risk map lists the answers to the questions, and provides the most likely diagnosis and level of risk for the diagnosis. The sensitivity for reaching diagnostic high risk thresholds can be changed. The care map provides evidence-based instructions on possible treatments and strategies for treatment changes should a particular treatment not work in a prescribed period of time, including what the patient should do, and what other treatments the physician should consider. The follow-up map provides data on symptom severity trends over time and the medical and personal history of the patient, as provided by the patient. The patient is encouraged to see his or her family physician to review the preliminary diagnostic risk and CARE MAPS with them.

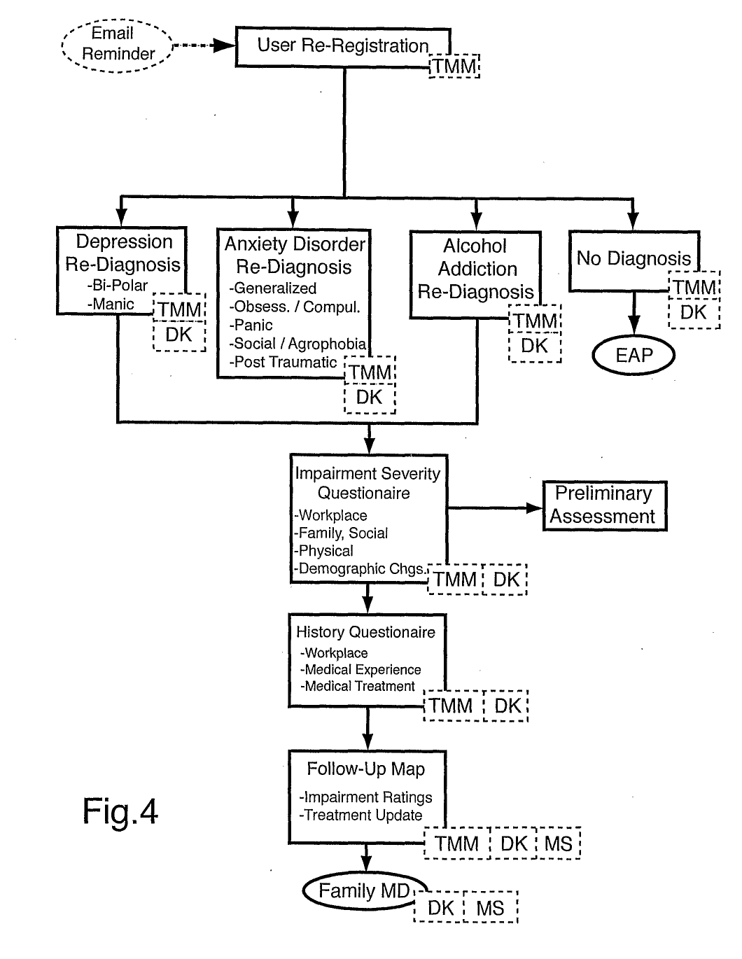

FIG. 4 illustrates the various stages when the patient makes a return visit to the system of the present invention. The patient is provided with an e-mail reminder which encourages the patient to visit the system's website every three weeks to reassess the significance of symptoms and answer four quality of life measures, specifically work life, home life, personal life and physical health. The patient is also asked to provide details about any medication taken and any psychotherapeutic treatment received.

Upon making a return visit to the website, the patient is asked to log in via his or her unique I.D. and password. The patient indicates whether he or she is following up, or merely reviewing his or her care status.

The patient is presented with the appropriate follow-up questions relating to his or her previously diagnostic risk symptoms and changes in work performance. These questions are identical to the ones in the original specific diagnostic risk module.

The patient is also asked questions regarding the management history of the disorder since their last assessment including physician visits, medication taken and psychotherapy received. The system provides a follow-up map including impairment ratings and a treatment update, and the patient is encouraged to print out and share this map with his or her family physician.

In addition, the system's website preferably provides access to a broad range of mental health information. For example, the website can provide hyperlinks to literature relating to the medical science behind mental health disorders in lay terms. Further, the system's website preferably provides hyperlinks to references to the most recent clinical and scientific literature at the medical professional level. Such links can boost compliance with “best practices” by both the patient and the physician.

Further, the system of the present invention preferably provides aggregate data which is collected regarding past psychiatric history and occupational information, including days off work, days missed, salary and demographic data including home life details. This type of data is crucial to providing return on investment information for an employer implementing the system of the present invention. It will also provide information concerning what treatments optimize return to work timing and/or decrease “presenteeism” (being at work but performing poorly).

Although this disclosure has described and illustrated preferred embodiments of the present invention, it is to be understood that the present invention is not restricted to these particular embodiments. Rather, the present invention includes all embodiments which are functional equivalents of the specific embodiments and features that have been described and illustrated herein. Many modifications and variations will now occur to those skilled in the art. For a definition of the present invention, reference is made to the following claims.

1. A method for treating a mental health disorder and improving workplace productivity and disability reduction related to said disorders comprising the steps of:
(a) having a patient access an interactive website for an initial visit;
(b) having the patient answer one or more screening questions to determine whether there is a possibility of the patient having a mental health disorder;
(c) processing the patient's answers to the one or more screening questions to determine whether there is the possibility of the patient having a mental health disorder;
(d) where there is the possibility of the patient having a mental health disorder, selecting a module of diagnostic risk assessment questions related to a specific mental health disorder;
(e) having the patient answer the diagnostic risk assessment questions related to the specific mental health disorder;
(f) processing the patient's answers to the diagnostic risk assessment questions to determine the level of risk for a particular diagnosis;
(g) providing the patient with a preliminary diagnostic risk assessment map which comprises a list of the patient's answers to the screening questions and the diagnostic risk assessment questions, and provides the level of risk for a particular diagnosis;
(h) providing the patient with a CARE MAP which comprises evidence-based instructions for patients and physicians on possible treatments;
(i) providing the patient with a follow-up map which comprises data on symptom and functional impairment severity trends over time, absenteeism, presenteeism, and medical and personal history of the patient, as provided by the patient;
(j) having the patient share the preliminary diagnostic risk assessment map, the CARE MAP and the follow-up map with a physician in printable form or via electronic records;
(k) after considering the preliminary diagnostic risk map, the CARE MAP and the follow-up map, the physician instituting medical treatment of the patient;
(l) providing the patient with a follow-up reminder via the internet and/or telephone to access the interactive website for a return visit;
(m) having the patient access the website for the return visit;
(n) having the patient answer the diagnostic risk questions from step (e) again;
(o) having the patient answer questions regarding management history of the medical treatment; and
(p) processing the patient's answers to the questions from steps (n) and (o) to determine whether there should be a change in the medical treatment of the patient.

2. The method according to claim 1, wherein the patient accesses the website anonymously.

3. The method according to claim 2, wherein the patient is provided with a user I.D. and password for accessing the website.

4. The method according to claim 1, further comprising the step of having the patient answer demographic questions.

5. The method according to claim 1, wherein the specific mental health disorder is selected from the group consisting of depression, bipolar disorder, post traumatic stress disorder, generalized anxiety disorder, panic anxiety disorder, obsessive-compulsive disorder, alcohol dependency, substance abuse, and eating disorders.

6. The method according to claim 1, wherein the website provides the patient with a physician's letter comprising a description of the purpose of the website and an overview of the website's credentials.

7. The method according to claim 1, wherein the follow-up reminder is provided three weeks after the patient's initial visit.

8. The method according to claim 1, wherein the website provides hyperlinks to literature on the clinical and/or medical science of mental health disorders.

9. The method according to claim 1, wherein said patients are employees and/or members of disability or health insurance plans.

10. The method according to claim 9, wherein the method is used in combination with an employee assistance program.

11. A system for treating mental health disorders and improving workplace productivity and disability reduction related to said disorders, wherein said system comprises an interactive website, said website comprising:
(a) a web page for accessing the web site;
(b) a screening questionnaire for determining whether there is a possibility of a patient having a mental health disorder;
(c) means for processing the answers to the screening questionnaire to determine whether there is the possibility of the patient having a mental health disorder;
(d) a diagnostic questionnaire for determining the patient's level of risk for a particular diagnosis;
(e) means for processing the answers to the diagnostic questionnaire to determine the patient's level of risk for a particular diagnosis;
(f) means for providing a preliminary diagnostic risk assessment map which comprises a list of the patient's answers to the screening questionnaire and the diagnostic risk assessment questionnaire, and provides the level of risk for a particular diagnosis;
(g) means for providing a CARE MAP which comprises evidence-based instructions for patients and physicians on possible treatments;
(h) means for providing a follow-up map which comprises data on symptom/functional impairment trends over time, and medical and personal history of the patient, as provided by the patient; and
(i) following medical treatment, means for providing a follow-up reminder via the Web and/or phone to the patient to access the website for a return visit.

12. The system according to claim 11, wherein the system provides means for the patient to access the website anonymously.

13. The system according to claim 11, wherein the website further comprises means for providing a physician's letter describing the purpose of the website and providing an overview of the website's credentials.

14. The system according to claim 11, wherein the website further comprises hyperlinks to literature on the clinical and/or medical science of mental health disorders.

15. A method for providing aggregate economic information to the employer/insurer regarding productivity losses due to mental disorders, most effective medical interventions in terms of restoration of productivity level and reduction of disability and aggregate information to public and private health insurers on most effective medical interventions that limit morbidity and mortality and promote early remission and recovery, said method comprising:
(a) having a patient access an interactive website for an initial visit;
(b) having the patient answer one or more screening questions to determine whether there is a possibility of the patient having a mental health disorder;
(c) processing the patient's answers to the one or more screening questions to determine whether there is the possibility of the patient having a mental health disorder;
(d) where there is the possibility of the patient having a mental health disorder, selecting a module of diagnostic risk assessment questions related to a specific mental health disorder;
(e) having the patient answer the diagnostic risk assessment questions related to the specific mental health disorder;
(f) processing the patient's answers to the diagnostic risk assessment questions to determine the level of risk for a particular diagnosis;
(g) providing the patient with a preliminary diagnostic risk assessment map which comprises a list of the patient's answers to the screening questions and the diagnostic risk assessment questions, and provides the level of risk for a particular diagnosis;
(h) providing the patient with a CARE MAP which comprises evidence-based instructions for patients and physicians on possible treatments;
(i) providing the patient with a follow-up map which comprises data on symptom and functional impairment severity trends over time, and medical and personal history of the patient, as provided by the patient;
(j) having the patient share the preliminary diagnostic risk assessment map, the CARE MAP and the follow-up map with a physician in printable form or via electronic records;
(k) after considering the preliminary diagnostic risk map, the CARE MAP and. the follow-up map, the physician instituting medical treatment of the patient;
(l) providing the patient with a follow-up reminder via the internet and/or telephone to access the interactive website for a return visit;
(m) having the patient access the website for the return visit;
(n) having the patient answer the diagnostic risk questions from step (e) again;
(o) having the patient answer questions regarding management history of the medical treatment;
(p) processing the patient's answers to the questions from steps (n) and (o) to determine whether there should be a change in the medical treatment of the patient; and
(q) having the patient answer questions about job classification, days absent, salary per day, days present but having percent productivity loss, days with symptoms, days of treatment (type and dosage), said data collected allowing correlation between clinical status, treatment, and workplace costs and savings (clinical economic correlation);
(r) processing data from (q) using analytical tools to derive clinico-economic information; and
(s) reporting to an entity arranging for access to the website with aggregate information regarding the value of workplace economic loss and benefit of the method.

16. The method according to claim 8, wherein said literature includes information on medical treatments for mental health disorders and the risks associated with terminating the medical treatments early.

17. The system according to claim 14, wherein said literature includes information on medical treatments for mental health disorders and the risks associated with terminating the medical treatments early.
